(19) United States
(12) Patent Application Publication
(10) Pub. No.: US 2022/0118045 A1
(43) Pub. Date: Apr. 21, 2022

(54) MEDICINAL PLANT PHYTOCHEMICAL EXTRACTS FOR COUGH REMEDY

(71) Applicant: ROGELIO TIMBOL SERAFICA, MAKATI (PH)

(72) Inventor: ROGELIO TIMBOL SERAFICA, MAKATI (PH)

(21) Appl. No.: 17/454,827

(22) Filed: Nov. 15, 2021

(51) Int. Cl. A61K 36/9068; A61K 36/61; A61K 36/9066; A61K 36/47; A61K 36/19; A61K 36/752

(52) U.S. Cl. CPC A61K 36/9068; A61K 36/61; A61K 36/752; A61K 36/47; A61K 36/19; A61K 36/9066

(57) ABSTRACT

The subject invention refers to the process of extracting phytochemicals and mixing of their active material composition from leaves, roots and fruits of several plants to formulate a cough syrup for the relief and treatment of mild & severe respiratory disorders brought about by bacteria and viruses, dust and pollens that may lead to difficulty in breathing resulting among others to coughs, allergies, colds, nasal congestions, pharyngitis, laryngitis, asthma and sneezing. The efficacy of the mixture of tropical plants having polyphenolic flavonoidal compounds derived from the basic below formula has yielded an effective remedy with little known side effects. I claim the formulations referred to as follows, but not limited thereto: Psidium guajava 25%, Zingiber officinale 30%, Curcuma longa 20%, Euphorbia hirta 10%, Adhatoda vasica 5%, and Citrofortunella microcarpa 3%.

### BACKGROUND OF INVENTION

With the advent of advancement in chemistry and in the field of chemical engineering and the proliferation of synthetic drugs due to the need to mass produce the much needed medicines, there had been very little focus and attention on the propagation and development of herbal medicines due to its nature of uncertainty brought about by climate change and natural problems encountered in agriculture, hence, herbal or medicinal plants which are the basic elements of the medicinal value and commerciality of tropical plant based medicines have been severely negatively affected.

However, since the ancient times, intrinsic and more acceptable use of plants since the ancient Hellenistic times from the time of the Greek five-volume book titled “De Materia Medica” originally written by Pedanius Dioscorides around 1^{st} Century A.D. who recorded several plants as a pharmacopeia and other men of medicine who painstakingly recorded several plants have been the very foundation of the present-day modern pharmacopeia. Some of the herbs and plants remain to this day as safe and tested and proven with little known side effects. Most of the herbs are known ingredients and are edible in small quantities. The combination of each herb which when put together becomes more potent and effective relief for most forms of respiratory or bronchial ailments and discomforts given the proper preparations and dosages.

The invention refers to the extraction of the various edible or medicinal plants' polyphenolic active materials that act as an effective medicine and form a synergistical combination that can deliver a remedy and a fast relief after taking moderate intake of the extracted tropical plant-based syrup as formulated. Inherent among the plant polyphenolic chemicals are known bactericidal and virucidal properties when combined synergistically either kill or destroy certain bacteria and viruses harmful to the body but leaving the healthy cells and friendly bacteria harmless¹. ¹Research Article—Rungsung W. Et Al. Secondary metabolites of plants in drug discovery. World Journal of Pharmaceutical Research, 2015; 4(7):604-13. The compound of one or two or several combinations react in the body as a protective and or ally of the human body's immune system against some form of respiratory ailments.

The efficacy of the herbal formula or combination herbs and certain percentages work out effectively as to provide quick relief against some respiratory ailments and symptoms like coughing, colds and difficulty in breathing. The formulations have an anti-tussive, anti-inflammatory/analgesic, mucolytic, bronchodilator and expectorant effects.

Based on the known efficacy and phytochemical components of each plant extract, medicinal ingredients and the required quantity where it is most effective, a safe and efficient bronchial or cough remedy can relieve and eliminate symptoms of bronchial ailments in a short period of time thereby giving the ailing person instant remedy and quick relief leading to a short duration of recovery and eliminate symptoms.

There are several synthetic medicines for different ailments but very few have several components of plant herbs for quick relief to aid some related bronchial ailments such as common colds, cough, asthma and allergy to dust or pollens and severe coughs. Most herb-based remedies or medicines are single based or two ingredients such as lemon and menthol, camphor and lime, or lemon with honey.

US Patent No.—0228383Ai composed of 17 main ingredients for cough syrup. Two known plant-based Turmeric Ginger (Curcuma longa) and Yellow Ginger (Zingiber officinale) are both ingredients commonly used for candies and seasoning were also used in the syrup. Adhatoda vasica has been in the market for decades which is present in the above claim. However, three more plant ingredients such as Guava (Psidium guajava), Asthma-Plant (Euphorbia hirta) and Citrofortunella microcarpa are not present in the formulations. Most of these plants are endemic to tropical countries most specially in Southeast Asia.

U.S. Pat. No. 6,287,567, herbal drink with different composition is entirely different from this invention.

U.S. Pat. No. 6,197,306, use of different herbs unrelated to pending patent.

U.S. Pat. No. 7,482,031 B2, Development of an anti-cough, anti-tussive and throat soothing herbal formulation.

In view of the need to provide different formulations from various tropical medicinal plants with traditional and clinical studies, these combination herbs have an effective efficacy for common respiratory ailments which are safe and reliable since all of the plants are either used for medicines, food, condiments and flavorings. The formulation is safe and efficacy is tested without harming the human body when taken in the dosage recommended.

All modes of extractions such as heating, drying, grinding and pulverizing have been utilized. The use of food and pharmaceutical grade additives and excipients have been added and the natural flavor of plants are enhanced for a more pleasant taste and easy to swallow elixir.

The syrup/elixir does not contain addicting or regulated drugs which may cause adverse effect on humans such as hallucination, dizziness, nor nausea and vomiting.

The syrup will be composed of derivatives of tropical herbs and fruits as main polyphenolic phytochemical ingredients. For excipients and preservatives to be safe for diabetic persons, sorbitol and artificial sugar and flavorings with natural lemony or menthol flavors are preferred.

The therapeutical elements would come from extracted polyphenolic materials of leaves, roots and fruit of several plants.

95% ethanolic solution derived from fruit plants and sodium benzoate are used to stabilize and preserve the concoctions respectively. Citrofortunella microcarpa fruit herb extracts, apart from its Vitamin C content or ascorbic acid their therapeutic values are used for soothing effect and sorbitol to sweeten the bitter concoction are used and to maintain the natural flavor and sweetness.

The herbal compound of the syrup would be composed of: Zingiber officinalis: Therapeutically used are roots. The roots contain essential oils 6-gingerol and shogaols which are known as anti-inflammatory properties. The rhizomes have all been traditionally used as a spice or as a food and refreshing drink as a tonic and for therapy whether for internal or external use². ²Research Article—Dried Ginger (Zingiber officinalis) Inhibits Inflammation in a Lipopolysaccharide-Induced Mouse Model; by You Yeon Choi, Mi Hye Kim, Jongki Hong, Sung-Hoon Kim, and WoongMoYang1; Received 21 Jan. 2013; Revised 5 Apr. 2013; Accepted 3 Jun. 2013; Hindawi Publishing Corporation.

Anti-inflammatory effects of Zingiber officinale: The extracts gingerols oils possessed the properties of its anti-inflammatory actions.

Zingiberaceae Curcuma longa: Therapeutically, the roots are the most sought after and prized property of Zingiberaceae Curcuma longa. The plant extract curcumin contains diferuloylmethane (curcumin1), desmethoxycurcumin (curcumin2) and bisdemethoxy curcumin curcumin (curcumin3). The three naturally curcumins composition I the extracts are natural known to inhibit inflammations due to bacterial or viral infections³. ³Review Article—Presenting a New Standard Drug Model for Turmeric and Its Prized Extract, Curcumin; by Franco Cavaleri; Received 27 Sep. 2017; Accepted 6 Dec. 2017; Published 15 Jan. 2018; Hindawi International Journal of Inflammation.

Euphorbia hirta: The leaves extract is traditionally used to treat asthma and carries quercetin compound, a flavonoid. This medicinal plant is endemic to Southeast Asian countries and more particularly widely used and propagated in the Philippines. It has been used widely against asthma, cough and bronchitis. It has a relaxant effect on the bronchial system. It is also being used against malaria and dengue with its flavanol glycoside properties⁴. ⁴Research Article —Biomimetic Synthesis of Silver Nanoparticles Using Endosymbiotic Bacterium Inhabiting Euphorbia hirta L. and Their Bactericidal Potential; by Baker Syed, 1 Hoovinakola Chinnappa Yashavantha Rao, Mysore Nagalingaswamy Nagendra-Prasad, Ashwini Prasad, Ballagere Puttaraju Harini, Pasha Azmath, Devaraju Rakshith, 1 and Sreedharamurthy Satish; Received 31 December 2015; Accepted 4 May 2016; Hindawi Publishing Corporation Scientifica. This is an open access article available for public and open to readers and researchers.

Adhatoda vasica: The leaves, roots, flowers and barks have therapeutic values. Essential oil from the plant and flavonoid quercetin is present from the leaves. The leaves extracts have been traditionally been used for treatment of respiratory ailments. For severe cough and hardened mucous the leaves are effective in easing the congestion⁵. ⁵Research Article—Antimicrobial, Antioxidant, and Cytotoxic Properties of Vasicine Acetate Synthesized from Vasicine Isolated from Adhatoda vasica L.; by V. Duraipandiyan, N. A. Al-Dhabi, 1 C. Balachandran, S. Ignacimuthu, C. Sankar, and K. Balakrishna2; Received 11 May 2014; Revised 28 Sep. 2014; Accepted 7 Oct. 2014; Hindawi Publishing Corporation BioMed Research International. This is an open access article available for public and open to readers and researchers.

Psidium guajava: The plant extract taken from the leaves of Psidium guajava have an antibacterial effect on bacteria specially on the inhibition of the growth of gram-positive bacteria cereus and Staphylococcus aureus a common bacteria that infects the throat and may cause itchy throat and coughing⁶.—⁶Research article “Antimicrobial Activities of Leaf Extracts of Guava [Psidium guajava L.] on Gram-Positive and Gram-Negative Bacteria by Bipul Biswas, Kimberly Rogers, Fredrick McLaughlin, Dwayne Daniels, and Anand Yadav, MS Biotechnology Program, College of Agriculture, Family Sciences and Technology, Fort Valley State University, Fort Valley, Ga. 31030, USA 2 Department of Biology, Fort Valley State University, Fort Valley, Ga. 31030, USA 3 Department of Chemistry, Fort Valley State University, Fort Valley, Ga. 31030, USA Correspondence should be addressed to Bipul Biswas; biswasb@fvsu.edu Received 4 Jul. 2013; Accepted 13 Sep. 2013 Academic Editor: Todd Callaway Copyright © 2013 Bipul Biswas et al. This is an open access article distributed under the Creative Commons Attribution License, which permits unrestricted use, distribution, and reproduction in any medium, provided the original work is properly cited. Received 4 Jul. 2013; Accepted 13 Sep. 2013.

Citrofortunella microcarpa: Or Calamansi is endemic in the Philippines and is equivalent to a lemon. The fruit is high in Vitamin C and it improves the immune system. It has an anti-inflammatory element due to presence of Tarmin in the rind. Due to high Vitamin C content of Citrofortunella microcarpa this is essential element in collagen production in the body necessary to build and repair tissue⁷.—⁷Research article: Judee N. Nogodula Et. Al. Phytochemical, antibacterial and mutagenic analyses of commercially available Calamansi, Citrofortunella microcarpa fruit extract, Phlippines E-journals, Chemistry, Pharmacology, pg 9, vol. 3, Nov. 1, 2012.

Dosage: Recommended dosage for cough syrup are as follows—for adults: 1 tablespoon every 4 hours and 3 times daily or as Physicians' advice. For children from 1 up to 6 years old: 1 teaspoon every 4 hours 3 times daily or as prescribed by a Physician.

### DETAILED DESCRIPTION OF INVENTION

The preparation of the extracted polyphenolic phytochemicals herbal mixture involves the sorting, cutting, washing, convection drying, filtering, pulverizing or grinding each herb specimen at the desired temperature and cleaving to achieve a fine powdered form in concentrate and the extract's component or ingredient for each herb. After the pulverized elements have been achieved to each desired mesh and size to make the solid particles easy to bind when mixed with aqueous or ethanolic solutions, the pulverized herbal components in powdered form shall then be mixed with purified water, xanthan gum and 95% ethanolic solutions, thereafter, sorbitol and other flavorings of tropical fruits and acceptable pharmaceutical grade preservative for longer stability and shelf life shall be added to make the syrup as a palatable cough remedy. The tropical herb compound formula will give instant respiratory and immediate relief to the patient arising from bacteria, viruses, dust and pollens resulting in mild to severe cough and colds, difficulty in breathing from allergies, asthma and sore throats.

### TECHNICAL DESCRIPTION

The subject invention refers to the extraction of active polyphenolic compound from tropical herbal composition: Psidium guajava 25%, Zingiber officinale 30%, Curcuma longa 20%, Euphorbia hirta 10%, Adhatoda vasica 5%, and Citrofortunella microcarpa 3%, for the relief and treatment of mild and severe coughs brought about by bacteria, viruses, dust and pollens that may lead to difficulty in breathing resulting among others in coughing, sneezing due to allergies, colds, nasal congestions, pharyngitis, laryngitis, asthma and other forms of bronchial, nasal and or certain respiratory ailments. The formulation shall result in an effective cough syrup remedy as an anti-tussive, anti-inflammatory, analgesic, bronchodilator, mucolytic and expectorant.

### Ingredients

Each 15 ml comprises of extracts derived from the following herbs:

| | | Active | Quantity | | |
|---|---|---|---|---|---|
| | Plant | Phytochemicals & | percentage | Quantity per | |
| Ingredients | Parts | Derivatives | per 15 ml | unit per 15 ml | |
| (1) Psidium Guajava | Leaves | Quercetin | 25 | 250 | mg |
| (2) Zingiber Officinale | Rhizome | Curcumin I, II, III | 30 | 300 | mg |
| (3) Curcuma Longa | Rhizome | Gingerols/Shogaols | 20 | 200 | mg |
| (4) Euphorbia Hirta | Leaves | Quercetin | 10 | 100 | mg |
| (5) Adhatoda Vasica | Leaves | Quercetin | 5 | 50 | mg |
| (6) Citrofortunella Microcarpa | Fruit | Citric Acid | 3 | 30 | mg |

### Process Flow to Obtain Plant Extracts' Polyphenolic Phytochemical and Other Derivatives

Sorting: The herb materials from leaves and rhizomes shall be sorted out as to remove impurities and non-essential plant parts. The leaves shall be freshly harvested and free from any pesticides or harmful chemicals. Rhizome parts shall be similarly be classified as to eliminate the presence of fungus or any other root crop pests or infestations.

Steaming and Boiling: The leaves of Psidium guajava shall be steamed in a steamer to soften plant tissue and to denature some flavonoids present in the herbs. This is also to reduce the amount of bitterness when used for the cough syrup from its powdered form. The Euphorbia hirta shall be washed and immersed in boiling or steamed water for 20 minutes as the leaves are thin and the liquid from the leaves contain the most essential ingredients which need to be extracted. Adhatoda vasica leaves shall be cleaned similarly with the Euphorbia hirta to be soaked 3 times in warm purified water.

The rhizomes of the Zingiber officinale and Curcuma longa shall be washed, scraped, brushed and cleaned with warm water to remove all dirt and impurities. Thereafter, rhizomes should be soaked in mild chlorinated water for two hours. Water should be changed twice after every two hours, thereafter, running water should remove the remaining dirt or impurities. The juice of the fruit of Citrofortunella microcarpa will be extracted by simple juicer equipment, thereby obtaining the main ingredient.

Cutting: The leaves shall be ready for drying and the rhizomes shall be cut into thin slices to reduce the size and in preparation for drying.

Drying (Artificial Drying in a Convection Oven): For the leaves' components, convection air dry for one hour at 40° C. until moisture content is reduced to a minimum of 90%. The rhizomes shall be convection dried at 50° C. for four hours until moisture content is reduced by 90%. For the leafy herbs convection drying shall result in a yellow dry leaf color at a 40° C. temperature and for at least one hour. The rhizomes, when dried, will turn into brownish color at four hours convection dried and 50° C. temperature.

Grinding and Pulverizing: After drying the leaves and rhizomes, the leaves shall be put in a grinding machine reduce in powdered form at least 297 μm (microns) or 0.297 mm mesh. After achieving the desired mesh, the powdered herbs shall be prepared for mixing.

Mixing: The pulverized herbs from the leaves and rhizomes shall be mixed in a stainless-steel cylinder together with the excipients, ethanol, sorbitol, sodium benzoate and Citrofortunella microcarpa flavoring and other inert ingredient to achieve a stable cough remedy solution ready for bottling in a 100 mL, 120 mL and 200 mL cough remedy pharmaceutical bottle.

### SUMMARY OF THE INVENTION

The claimed invention is on the discovery of the extraction and mixture of plants' and herbs' polyphenolic compounds when combined through same basic processes of extraction and mixing of certain parts of the plant and on the mixed proportion of ingredients by weight synergistically can provide relief and remedy for mild and severe cough that maybe due to bacteria, viruses, dust fungus, pollens, allergies and asthma and eliminate their symptoms thereof.

The syrup or elixir has a broad application that it can relieve, mild and severe cough, itchy throat, shortness of breathing and sore throat related to bacterial, fungal, viral and or allergies due to dust or pollens. Very little side effects are known since individually the polyphenolic compounds and their components are edible and non-toxic to humans and are used traditionally as medicines or food. The formulations have efficacies as antitussive, anti-inflammatory, analgesic, bronchodilator, mucolytic and expectorant.

The syrup does not contain regulated drugs like codeine or ephedrine which are known for serious side effects that may cause addictions.

The formulations of the compound's ingredients are all based on the therapeutic value of each plant and their active polyphenolic materials which have been extracted and are mixed into a cough syrup as follows:

| | Therapeutic | Active |
|---|---|---|
| Botanical Name: | Properties: | Phytochemicals |
| (1) Psidium Guajava | Antibacterial for gram | Quercetin |
| | positive bacteria cereus | |
| | and staphylococcus aureus | |
| (2) Curcuma Longa | Analgesic, Anti- | Curcumin I, II, III |
| | inflammatory | |
| (3) Zingiber Officinale | Anti-inflammatory, | Gingerols/Shogaols |
| | Antibacterial, Mucolytic | |
| (4) Euphorbia Hirta | Anti-asthma, | Quercetin |
| | Anti-allergy | |
| (5) Adhatoda Vasica | Anti-mucolytic or | Quercetin |
| | Expectorant, | |
| | Bronchodilator | |
| (6) Citrofortunella Microcarpa | Anti-inflammatory, | Citric Acid |
| | Bronchodilator, | |
| | Expectorant | |

The extracted active polyphenolic materials and mixtures from various tropical herbs from leaves, fruits and roots provide instant relief for some respiratory ailments and act as antitussive, anti-inflammatory, analgesic, bronchodilator, mucolytic and expectorant.

Examples 1 to 5 of the ingredients per 15 ml or one tablespoon. Formulations claimed from tropical plants extracts for every 15 ml are composed of the following elements and materials:

Example 1

| Botanical Name | Qty in % |
|---|---|
| Psidium Guajava | 24.73 |
| Zingiber Officinale | 30.22 |
| Curcuma Longa | 20.25 |
| Euphorbia Hirta | 9.8 |
| Adhatoda Vasica | 5.35 |
| Citrofotunella Microcarpa | 3.2 |

Compositions of the tropical plant extracts as in claim 1 have the following elements and materials for every 15 ml.

Example 2

| Botanical Name | Qty in % |
|---|---|
| Psidium Guajava | 10.23 |
| Zingiber Officinale | 24.89 |
| Curcuma Longa | 19.81 |
| Euphorbia Hirta | 30.20 |
| Adhatoda Vasica | 3.45 |
| Citrofotunella Microcarpa | 4.32 |

Compositions of the tropical plant extracts as in claim 1 have the following elements and materials for every 15 ml.

Example 3

| Botanical Name | Qty in % |
|---|---|
| Psidium Guajava | 14.97 |
| Zingiber Officinale | 25.30 |
| Curcuma Longa | 4.79 |
| Euphorbia Hirta | 20.32 |
| Adhatoda Vasica | 4.89 |
| Citrofotunella Microcarpa | 15.23 |

Compositions of the tropical plant extracts as in claim 1 have the following elements and materials for every 15 ml.

Example 4

| Botanical Name | Qty in % |
|---|---|
| Psidium Guajava | 29.69 |
| Zingiber Officinale | 20.14 |
| Curcuma Longa | 14.97 |
| Euphorbia Hirta | 10.46 |
| Adhatoda Vasica | 4.89 |
| Citrofotunella Microcarpa | 9.98 |

Compositions of the tropical plant extracts as in claim 1 have the following elements and materials for every 15 ml.

Example 5

| Botanical Name | Qty in % |
|---|---|
| Psidium Guajava | 15.27 |
| Zingiber Officinale | 19.83 |
| Curcuma Longa | 20.22 |
| Euphorbia Hirta | 10.26 |
| Adhatoda Vasica | 4.89 |
| Citrofotunella Microcarpa | 14.88 |

Compositions of the tropical plant extracts as in claim 1 have the following elements and materials for every 15 ml.

Inert Ingredients and excipients such as ethanol, sorbitol, flavorings and preservatives will remain constant for every 15 ml dosage on all the seven examples and on claim 1 as follows:

Excipients and inert ingredients per 15 ml dose:

| | | |
|---|---|---|
| Excipient | Qty | % |

| | | | |
|---|---|---|---|
| 1. Sorbitol | 600 | mg | 26 |
| 2. Ethanol 95% | 1500 | mg | 65 |
| 3. Sodium Benzoate | 10 | mg | .40 |
| 4. Glycerin | 100 | mg | 4.3 |
| 5. Flavorants | 100 | mg | 4.3 |

| | |
|---|---|
| 6. Purified Water | q.s |

| | | | |
|---|---|---|---|
| Total Excipients & Inert | 2,310 | mg | 100 |
| Ingr. | | | |
| (Excluding purified water) | | | |

The combinations of the efficacy of the syrup of the subject invention shall be adjusted for applications on different levels of target ailments based on the following examples. The main ingredients may be increased or decreased depending on the application of the invention such as formulations for dry cough, too much phlegm, sneezing, clogged nose or runny nose, whooping cough and itchy throat.

Therefore, different amounts of the main active ingredients shall be subject to adjustments. To blend the ingredients into a syrup form, excipients shall be added such as ethanol, glycerin and sorbitol. Optional ingredients maybe added for flavoring aromatics and sweetener to tone down the bitter taste of the syrup. The excipients preferred are sucrose, honey, sorbitol or coconut-based sugar. For added flavorings, methyl salicylate, peppermint, lemon grass or Pandan leaves. Sodium Benzoate as preservative is more preferred. Other miscellaneous excipients maybe added.

1. I claim the formulations referred to as follows, but not limited thereto.

| | Quantity |
|---|---|
| Botanical Name: | in % |
| 1. Psidium Guajava | 25 |
| 2. Zingiber Officinale | 30 |
| 3. Curcuma Longa | 20 |
| 4. Euphorbia Hirta | 10 |
| 5. Adhatoda Vasica | 5 |
| 6. Citrofortunella Microcarpa | 3 |

2. The discovery of a mixture of various polyphenolic phytochemical from plants when combined through some basic processes of extraction of certain parts of the plant and on the mixed proportion of ingredients by weight can provide relief remedy for mild and severe cough that maybe due to bacteria, viruses, dust, fungus, pollens, allergies and asthma and minimize if not eliminate their symptoms altogether.

3. The syrup or elixir has a broad application that it can relieve, mild and severe cough, itchy throat, shortness of breathing and sore throat related to bacterial, fungal, viral and or allergies due to dust or pollens. Very little side effects are known since individually the compounds and their polyphenolic phytochemicals and some derivatives components are edible and non-toxic to humans and are used traditionally as medicines. The formulations have efficacies as antitussive, anti-inflammatory, analgesic, bronchodilator, mucolytic and expectorant.
